(19) United States
(12) Patent Application Publication
(10) Pub. No.: US 2006/0264646 A1
(43) Pub. Date: Nov. 23, 2006

(54) Transformation of tricyclopentabenzene (trindane) to 12 hydroxy - 16 - oxatetracyclo [10.3. 1.01.5.07.11] hexadec - 7(11) - EN -2,6- dione

(75) Inventors: Ranganathan Subramania, Hyderabad (IN); Manjheri Muraleedharan Kannoth, Hyderabad (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 11/409,578

(22) Filed: Apr. 24, 2006

Related U.S. Application Data
(63) Continuation of application No. 10/702,233, filed on Nov. 6, 2003, now abandoned.
Application No. 10/702,233 is a continuation of application No. 09/813,111, filed on Mar. 20, 2001, now abandoned.

(51) Int. Cl. C07D 311/78; C07D 493/04

(52) U.S. Cl. 549/383

(57) ABSTRACT

The present invention relates to a process for the transformation of tricyclopentabenzene to 12-hydroxy-16-oxatetracyclo[10.3.1.0^{1,5l .0}^{7.11}]hexadec-7(11)-en-2,6-dione of formula 2 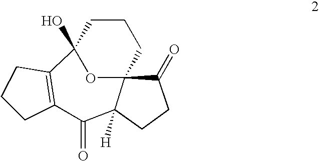
by ozonolysing two out of three double bonds of trindane, followed by aldol condensation and adding the hydroxyl group to the carbonyl function intramolecularly to obtain 12-hydroxy-16-oxatetracyclo[10.3.1.0^{1,5}.0^{7,11}]hexadec-7(11)-en-2,6-dione.

### FIELD OF THE INVENTION

The present invention relates to the transformation of tricyclopentabenzene (trindane) to 12-hydroxy-16-oxatetracyclo[10.3.1.0^{1,5}.0^{7,11}]hexadec-7(11)-en-2,6dione of the formula 2, an important natural product analogue.

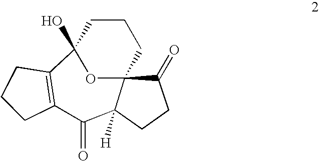

### BACKGROUND OF THE INVENTION

The development of novel and shortest synthetic routes towards natural products and their analogs having immense importance as drug candidates continues to be a challenge in organic chemistry. Although many interesting synthetic strategies have been developed towards this (eg. M. H. Jose Ignacio, R. F. Maria del Rosario, C. L. Jose Ignacio, Nicolas Birlirakis, and Simeon Arseniyadis, Tet. Asymm. 2000, 11, 4, 951-973; Gary A. Sulikowski, Fabio Agnelli, and R. Michael Carbett, J. Org. Chem, 2000, 65, 337-342 and the references therein), the main drawback in many cases is the large number of reaction steps and relatively poor yields of the target molecule. Many reagents employed in such synthesis are costly which makes the strategy less economic.

Earlier efforts towards the synthesis of clathrin models (E. Ungewickell, Current Biol., 1999, 9, 1, R32-35) has resulted in the RuVIII mediated transformation of trindane to 4-[(1R,2S,4R,5S)-1,2,5-trihydroxy-3-oxabicyclo[3.3.0]octane-4 spiro-1′-(2′-oxocyclopentan)-2-yl] butanoic acid (S. Ranganathan, K. M. Muraleedharan, P. Bharadwaj, and K. P. Madhusudanan, J. Chem. Soc. Chem. Commun., 1998, 2239-2240). The exclusive π oxidation observed here at once suggested a high reactivity for the double bonds, which is confined within the closed framework of peripheral methylenes.

Accordingly, it is important to develop a process for the development of synthetic routes for natural product analogues that is cheap and at the same time provides a high yield.

### OBJECTS OF THE INVENTION

The main objective of the present invention is to transform trindane (1), a readily available hydrocarbon to a highly functionalized and condensed tetracyclic system 2 having structural resemblance to natural products (J. S. Clark, A. G. Dossetter. A. J. Blake, W. S. Li, and W. G. Whittingham, J. Chem. Soc. Chem. Commun., 1999. 749-750: J. S. Clark and Y. S. Wong, J. Chem. Soc. Chem. Commun., 1999, 1079-1080), in one step by ozonolysis.

It is another objective of the invention to provide a process for the synthetic production of natural product analogues that is cheap and still results in high yield.

It is a further objective of the invention to provide a synthetic route for the production of natural product analogues that overcomes the drawbacks associated with the prior art above.

### SUMMARY OF THE INVENTION

Accordingly, the present invention relates to a process for the transformation of tricyclopentabenzene (trindane) to 12-hydroxy-16-oxatetracyclo[10.3.1.0^{1,5}.0^{7,11}]hexadec-7(11)-en-2,6-dione of the formula 2, 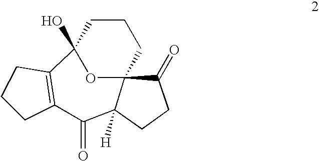
said process comprising ozonolysing two out of three double bonds of trindane, followed by aldol condensation and adding the hydroxyl group to the carbonyl function intramolecularly to obtain 12-hydroxy-16-oxatetracyclo[10.3.1.0^{1,5}0^{7,11}]hexadec-7(11)-en-2,6-dione of formula 2.

In one embodiment of the invention, the ozonolysis of tricyclopentabenzene is done by passing ozonised oxygen through a solution of tricyclopentabenzene in dry CH₂Cl₂ admixed with dimethyl sulfide.

In another embodiment of the invention, ozonolysis is done at a temperature in the range of −70 to −80° C.

In a further embodiment of the invention the reaction is carried out as a one pot reaction.

### DETAILED DESCRIPTION OF THE INVENTION

Earlier efforts towards the synthesis of clathrin models (E. Ungewickell, Current Biol., 1999, 9, 1 R32-35) has resulted in the RuVIII mediated transformation of trindane to 4-[(1R,2S,4,R,5S)-1,2,5-trihydroxy-3-oxabicyclo[3.3.0]octane-4 spiro-1′-(2′-oxocyclopentan)-2-yl] butanoic acid (S. Ranganathan, K. M. Muraleedharan, P. Bharadwaj and K. P. Madhusudanan, J. Chem. Soc. Commun., 1998, 2239-2240). The exclusive π oxidation observed here indicated a high reactivity for the double bonds, which is confined within the closed framework of peripheral methylenes. As a result, the reactivity of the aromatic π system in trindane towards ozone was studied.

The synthetic route for the preparation of 12-hydroxy-16-oxatetracyclo[10.3.1.0^{1,5}.0^{7,11}]hexadec-7(11)-en-2,6-dione (2) by the ozonolysis of trindane (1) is given below and scheme 1 represents the mechanism for this transformation.

Synthetic route for the preparation 12-hydroxy-16-oxatetracyclo[10.3.1.0^{1,5}.0^{7,11}]hexadec-7(11)-en-2,6-dione (2)

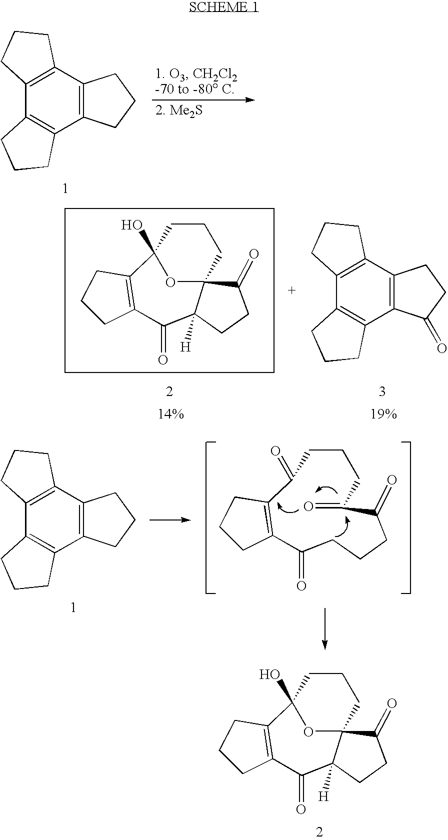

The following example is given by way of illustration only and therefore should not be construed to limit the scope of the present invention.

### EXAMPLE

### Ozonolysis of Trindane: Isolation of 12-hydroxy-16-oxatetracyclo[10.3.1.0^{1,5}.0^{7,11}]hexadec-7(11)-en-2,6-dione (2)

Ozonized oxygen was bubbled through a solution of Trindane (3.1 g. 15.65 mmol) in dry CH₂Cl₂ (150 mL) at ˜−70 to −80° C. for 2.5 h, admixed with Dimethyl sulfide (5.2 mL), left stirred for 2 h, treated with saturated NaHCO₃ (20 mL), and stirred for an additional 1 h. The organic layer was separated and the aqueous layer washed with additional CH₂Cl₂ (3×25 mL). The organic layers were combined, washed with distilled water (1×10 mL), dried (MgSO₄), evaporated under vacuo and the residue chromatographed on silica gel. Elution with hexane-EtOAc (2:1) afforded 2 as a crystalline solid. The reaction in addition gave 19% yield of monobenzylic oxidation product of trindane (3), together with 2 g of unreacted trindane. The percentage yields of the products were calculated based on the amount of trindane reacted.

2) Yield: 0.2 g (14%); Mp.: 138-140° C.; IR (neat):3400 (br), 2944, 1752, 1680, 1440, 1040; ¹H NMR (CDCl₃) δ 1.38-2.2 (m, CH₂—CH₂—C=C,CH₂—CH₂—C=O & CH₂ of pyran ring), 2.3-3.0(m,—CH₂—C=O,2×CH₂—C=C), 3.23 (m,1H,—(CO)—CH(CH₂)₂—C=O); ¹³C NMR (CDCl₃) δ 17.93-35.3(8×CH₂),58.36(—CH),81.20 (quaternary carbon) 98.50 (C—OH), 140.84, 154.60 (—C=C—), 198.58 (C=O, conjugated), 212.96 (C=O, non-conjugated) FAB MS (m/z) (%):263 (56%) (MH)⁺, 285 (30%) (M+Na)⁺, EI MS: 262.

The proposed structure of this compound has been confirmed by X-ray crystallography. 3) Yield: 0.23g (19%); IR (neat): 3424 (br, enolization), 2944, 1712, 1600, 1400, 1272, 1120; ¹H NMR (CDCl₃) δ 2.19(m,4H2×CH₂), 2.64-3.00(m,10H, benzylic CH₂s), 3.2 (t,2H,CH₂—CO); ¹³C NMR (CDCl₃) δ 24.52-36.82(8×CH₂), 139.1-149.4(6×C aromatic), 207.67(C=O); FAB MS (m/z) (%):213 (100%) (MH)⁺

The main advantages of the present invention are

1. The present compound, 12-hydroxy-16-oxatetracyclo[10.3.1.0^{1,5}.0^{7,11}]hexadec-7(11)-en-2,6-dione, a potentially important natural product analogue (2) which is highly functionalized and condensed tetracyclic system, is synthesized in one step.

2. The starting material trindane is a readily available hydrocarbon which makes the synthesis more economic.

3. The only reagents used to effect the transformation are ozone and dimethyl sulfide, which also makes the synthesis more economic.

4. The reaction is a one-pot reaction without requiring isolation of intermediates, making the process more convenient.

5. The various functional groups present in 2 (ie. hydroxyl, carbonyl and olefinic) could be used for its transformation to various structural analogs or other natural products of therapeutic importance.

We claim:

1. A process for the transformation of tricyclopentabenzene to 12hydroxy-16-oxatetracyclo[10.3.1.0^{1,5}.0^{7,11]}hexadec-7(11)-en-2,6-dione of the formula 2 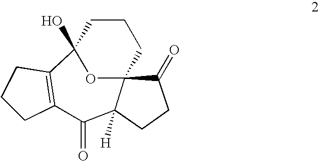
said process comprising ozonolysing two out of three double bonds of trindane, followed by aldol condensation and adding the hydroxyl group to the carbonyl function intramolecularly to obtain 12-hydroxy-16-oxatetracyclo[10.3.1.0^{1,5}.0^{7,11}]hexadec-7(11)-en-2,6-dione of formula 2.

2. A process as claimed in claim 1 wherein the ozonolysis of tricyclopentabenzene is done by passing ozonised oxygen through a solution of tricyclopentabenzene in dry CH₂Cl₂ admixed with dimethyl sulfide.

3. A process as claimed in claim 2 wherein ozonolysis is done at a temperature in the range of −70 to −80° C.

4. A process as claimed in claim 1 which is a one pot reaction.
